(19) United States
(12) Patent Application Publication
(10) Pub. No.: US 2016/0128943 A1
(43) Pub. Date: May 12, 2016

(54) CONTROLLED RELEASE CAFFEINE DOSAGE FORMS

(71) Applicant: KVK-TECH, INC., Newtown, PA (US)

(72) Inventors: Kalyan Vepuri, Newtown, PA (US); Narender R. Uppugalla, Hauppauge, NY (US)

(73) Assignee: KVK-TECH, INC., Newton, PA (US)

(21) Appl. No.: 14/896,004

(22) PCT Filed: Jun. 4, 2014
(86) PCT No.: PCT/US2014/040916

§ 371 Date: Dec. 4, 2015

Related U.S. Application Data
(60) Provisional application No. 61/830,830, filed on Jun. 4, 2013.

(51) Int. Cl. A61K 9/20; A61K 9/28; A61K 9/24; A61K 31/519

(52) U.S. Cl. CPC A61K 9/2054; A61K 31/519; A61K 9/2846; A61K 9/2027; A61K 9/209; A61K 9/2866; A61K 9/2853

(57) ABSTRACT

Formulations capable of extended or sustained release of high levels of caffeine or analogs, derivatives and metabolites thereof have been developed The formulations contain at least two components capable of releasing the caffeine or related compound differing rates to maintain a desired plasma level.

### CROSS-REFERENCE TO RELATED APPLICATIONS

This application claims the benefit of U.S. Provisional Application No. 61/830,830, filed on Jun. 4, 2013.

### FIELD OF THE INVENTION

The present invention relates to orally administered formulations of caffeine and related compounds. The formulations are capable of delivering an immediate release bolus of drug followed by an extended release dose of the drug. The formulations provide useful in vivo blood plasma concentrations of caffeine and related compounds over extended periods of time.

### BACKGROUND OF THE INVENTION

Caffeine is an alkaloid having the chemical formula C₈H₁₀N₄O₂. It belongs to the family of chemicals known as methylxanthines, which also includes the closely related chemicals theophylline and theobromine In its pure form, caffeine is an odorless, white solid which can be in the form of fleecy masses, glistening needles or powder.

Caffeine and the other methylxanthines are found naturally in plants. Tea, which is prepared from the leaves of the plant Thea sunensis, naturally contains all three of the aforementioned methylxanthines and is consumed by at least half of the world population. Cocoa and chocolate are produced from the seeds of Theobroma cacao and contain both caffeine and theobromine The most obvious and important source of American caffeine intake, coffee, is produced from the Coffea arabica plant. Prior to the deliberate insertion of additional caffeine during production, many sodas contain a natural form of caffeine obtained from extracts of the nuts of Cola acuminata. While it occurs abundantly in nature from a wide variety of sources, caffeine is also created synthetically and by extraction from cocoa, coffee bean or tea leaf waste, which allows for its inclusion in a greater variety of consumer products.

Caffeine has a variety of pharmacological effects on organ systems and neural functions, though the level and duration of the effect varies among individuals. It is absorbed into the bloodstream following ingestion via the lining of the stomach and the small intestine, and reaches peak levels in the circulation of the bloodstream between fifteen and forty-five minutes after consumption. Caffeine stimulates the central nervous system, reaching its maximum effect between thirty and sixty minutes after absorption; this is accompanied by a temporary increase in metabolic function.

At least one product containing caffeine as the sole active ingredient has been approved by the FDA. Caffeine citrate is marketed as an injectable solution under the name CAFCIT® and is approved for the short-term treatment of apnea of prematurity in infants.

Caffeine has long been employed medically as a mild diuretic. Caffeine also acts as a stimulant for the cardiovascular system, though the actions of the methylxanthines on the circulatory system are complex and sometimes antagonistic, and the resulting effects largely depend on the conditions prevailing at the time of their administration. Higher concentrations of caffeine have been known to produce tachycardia and other cardiac arrhythmias, but the risk of this in normal healthy individuals is minimal. These pharmacological effects last only as long as caffeine remains in the bloodstream; as time progresses following ingestion and absorption, the liver metabolizes caffeine. It is then excreted from the body through a number of channels, including urine, saliva, semen, and even breast milk. While a number of factors, among which are pregnancy, liver disease, body weight, concurrent medications, and natural metabolic rate all influence the body's ability to break down caffeine, its average half-life is three and one half hours, meaning that the average person will eliminate half of the amount of ingested caffeine within that time span.

The market for products delivering high levels of caffeine over a prolonged period of time has exploded in recent years. However, caffeine, despite being a natural product, is regulated by the Food and Drug Administration when it is provided at high levels, due to the risks noted above. How the U.S. Food and Drug Administration (FDA) treats a product with caffeine in it depends on whether or not it is considered a food or a drug. Caffeine, when categorized as a food, is fit for human consumption and is generally recognized as safe. Under 21 Code of Federal Regulations Section 182.1180, the federal government states that caffeine is generally recognized as safe as used in cola or soft-drink products and when it is used in accordance with proper manufacturing processes. Safe substances do not require any FDA approval as long as they fall within the safe levels dictated by the statute. Any product manufactured with caffeine must have 0.02 percent or less of the substance in the product to be considered safe.

Caffeine content can differ markedly even within a product category, for example, the amount of caffeine present in “real-world coffee” can range from seventy-five to two-hundred-fifty milligrams per serving. When caffeine is used as a drug, such as in a diet pill or other product such as some over the counter migraine remedies, the FDA has a strict approval process through which a manufacturer must proceed before the drug is approved for sale in the United States. Most available formulations contain 200 mg or less caffeine to avoid regulatory restrictions.

Numerous formulations have been developed which allegedly provide a controlled release of caffeine. The formulations typically contain a controlled release mechanism, such as a matrix, semipermeable coating, osmotic system, or controlled particle size. Many of these formulations are enterically coated to delay release after ingestion, until the formulation enters the small intestine. Others are microencapsulated.

Delayed release formulations are difficult to prepare because caffeine is a small and water soluble molecule. Most formulations only contain 100 to 200 mg caffeine, and do not provide truly effective in vivo concentrations of caffeine over prolonged time periods. Because they lack sufficiently controlled sustained release, formulations containing more than 200 mg caffeine may produce unacceptably high concentrations of caffeine in the initial burst, and thus will not meet FDA requirements for safety.

None of these products are designed to provide controlled, sustained, uniform levels of caffeine. Most formulations either have a burst effect, with rapidly declining levels over time, or only slowly raise caffeine levels over time and do not reach desired plasma concentrations.

There is a need for a product which produces a “loading dose” of caffeine, i.e., an initial immediate release of caffeine, followed by a controlled release of product to maintain the levels over time in the body as the caffeine is metabolized and excreted.

It is an object of the present invention to provide a caffeine formulation having an initial immediate release of caffeine, followed by a controlled release of product to maintain the levels over time in the body as the caffeine is metabolized and excreted.

It is a further object of the invention to provide an orally available delayed release formulation of caffeine containing higher amounts of caffeine than heretofore available.

### SUMMARY OF THE INVENTION

A caffeine-containing dosage form has been developed which provides an initial immediate release of caffeine, followed by a controlled release of caffeine to maintain caffeine levels in the body over time. Generally, the dosage form contains at least one component capable of producing a rapid rise in plasma caffeine concentration, and at least one component capable of releasing caffeine over time sufficient to maintain plasma caffeine concentrations over time as the initially released caffeine is metabolized. Generally, the formulation immediately releases a caffeine bolus followed by a sustained and/or delayed release of caffeine to maintain an in vivo blood plasma concentration of caffeine between 0.3 micrograms/mL and 40 micrograms/mL.

### BRIEF DESCRIPTION OF THE DRAWINGS

FIG. 1 is a graph depicting the release of caffeine (% caffeine released) from exemplary formulation 98A/E and 104 A/E as a function of time (hours).

FIG. 2 is a graph depicting the release of caffeine (% caffeine released) from other exemplary formulations 52-132D/F, 52-145 C/D, and 52-180 D, as a function of time (hours). The dissolution test was performed in 0.1 N HCl for two hours, in 5.5 acetate buffer between 2-3 hours and 3-8 hours in 6.8 phosphate buffer

### DETAILED DESCRIPTION OF THE INVENTION

### I. DEFINITIONS

As used herein, the term “formulation” refers to dosage units containing at least one physiologically active compound and one of more pharmaceutically acceptable excipients.

As used herein, the term “oral formulation” refers to dosage units which may be administered to a patient by mouth. Exemplary oral formulations include tablets, capsules and pills.

The term “immediate release” (IR) refers to release of an active agent to an environment over a period of seconds to up to about 30 minutes from initiation of release, wherein release begins no more than about 10 minutes after exposure to an aqueous environment. An immediate release composition, which does not possess a substantial delay in drug release, should be considered as a subset of a rapid release composition. An immediate release composition releases drug in the buccal cavity, esophagus and/or stomach.

“Rapid release” as used herein refers to release of an active agent to an environment over a period of seconds to no more than about 60 minutes once release has begun and release can begin within a few seconds or minutes after exposure to an aqueous environment or after completion of a delay period (lag time) after exposure to an aqueous environment. Overall, a rapid release composition releases drug in the stomach, jejunum or duodenum after oral administration, provided the composition does not include a delayed release material or delayed release coating. In such case, the rapid release composition would release drug in the intestine high, medium and/or low or colon depending upon the nature of the delayed release polymer coating.

As used herein, “controlled release” refers to a release profile of a drug for which the drug release characteristics of time course and/or location are chosen to accomplish therapeutic or convenience objectives not offered by conventional dosage forms such as solutions, or immediate release dosage forms. Delayed release, extended release, sustained release and their combinations are types of controlled release.

In preferred formulations a combination of extended release components, rapid release components, immediate release components, and delayed release components are combined to provide caffeine dosage forms having the desired release profile and/or pharmacokinetic parameters.

As used herein, the term “bolus” refers to an amount of caffeine or other drug that is delivered in an immediate or rapid release dosage form. The delivery of a bolus of caffeine or other drug cause a rapid rise in the blood plasma concentration of the caffeine or other drug.

As used herein, the term “release controlling polymer” refers to a pharmaceutically acceptable polymeric compound which impacts the release rate of a drug from a dosage form.

Throughout the disclosure reference is made to various units of mass, time, size, and plasma concentrations. One of ordinary skill will appreciate that the precision of an individual measurement is dependent on the feature being assayed and the measurement tool or method employed. As used herein, the term “about” refers to the variance that one of ordinary skill would expect from measurements in which absolute precision is not possible.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable salt,” as used herein, refer to ionic derivatives of the compounds defined herein, wherein the parent compound is modified by reaction with a suitable acid or base. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, hydroiodic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

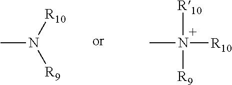

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In still more preferred embodiments, the term “amine” does not encompass amides, e.g., wherein one of R₉ and R₁₀ represents a carbonyl. In even more preferred embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

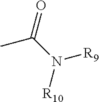

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

The term “carbonyl” is art-recognized and includes such moieties as can be represented by the general formula:

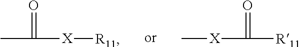

wherein X is a bond or represents an oxygen or a sulfur, and R11 represents a hydrogen, an alkyl, a cycloalkyl, an alkenyl, an cycloalkenyl, or an alkynyl, R′11 represents a hydrogen, an alkyl, a cycloalkyl, an alkenyl, an cycloalkenyl, or an alkynyl. Where X is an oxygen and R11 or R′11 is not hydrogen, the formula represents an “ester”. Where X is an oxygen and R11 is as defined above, the moiety is referred to herein as a carboxyl group, and particularly when R11 is a hydrogen, the formula represents a “carboxylic acid”. Where X is an oxygen and R′11 is hydrogen, the formula represents a “formate”. In general, where the oxygen atom of the above formula is replaced by sulfur, the formula represents a “thiocarbonyl” group. Where X is a sulfur and R11 or R′11 is not hydrogen, the formula represents a “thioester.” Where X is a sulfur and R11 is hydrogen, the formula represents a “thiocarboxylic acid.” Where X is a sulfur and R′11 is hydrogen, the formula represents a “thioformate.” On the other hand, where X is a bond, and R11 is not hydrogen, the above formula represents a “ketone” group. Where X is a bond, and R11 is hydrogen, the above formula represents an “aldehyde” group.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C1-C10) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heteroatom” as used herein means an atom of any element other than carbon or hydrogen. Preferred heteroatoms are boron, nitrogen, oxygen, phosphorus, sulfur and selenium. Other heteroatoms include silicon and arsenic.

### II. COMPOSITIONS

A. Caffeine

The formulations contain caffeine or a pharmaceutically acceptable salt, analog, derivative, or metabolite thereof. The structure of caffeine is shown below:

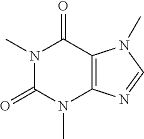

The term “analog”, as used herein refers to a compound in which one or more atoms are replaced with a different atom or group of atoms. The term “analog” can generally refer to a compound that resembles another in structure but is not necessarily an isomer, for example, the term “analog” can be understood to encompass a substance which does not contain the same basic carbon skeleton and carbon functionality in its structure as a “given compound”, but which can mimic the given compound by incorporating one or more appropriate substitutions such as for example substituting carbon for heteroatoms.

The term “derivative”, as used herein, refers to a compound that is derived from a parent compound or a compound that can be imagined to arise from the parent compound via one or more chemical modifications. The term “derivative” refers to a compound that at least theoretically can be formed from the precursor compound. The term “derivative” encompasses salts, hydrates, protected forms, esters, amides, active metabolites, of the parent compound, preferably those which are not biologically or otherwise undesirable and induce the desired pharmacological and/or physiological effect. Preferably the derivative is pharmaceutically acceptable. In some embodiments, the term derivative does not encompass naturally-occurring or native compounds.

A non-limiting list of caffeine analogs and derivatives that can be used include, but are not limited to, xanthine, hypoxanthine (6-hydroxypurine), 1-methylxanthine, 3-methylxanthine, 7-methylxanthine, azaxanthine (8-aza-2,6-dihydroxypurine), theophylline, theobromine, 3,7-dimethyl-1-propargylxanthine, 1,3-dipropyl-7-methylxanthine.

In some embodiments, the formulations can contain 3,7-dimethyl-1-propargylxanthine having the structure depicted below.

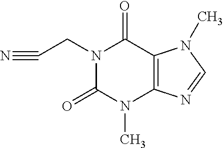 [SMILES: Cn1cnc2c1c(=O)n(CC#N)c(=O)n2C]

In some embodiments, the formulations can contain 1,3-dipropyl-7-methylxanthine having the structure depicted below.

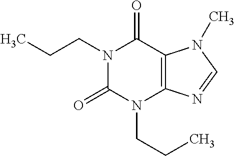 [SMILES: CCCn1c(=O)c2c(ncn2C)n(CCC)c1=O]

In some embodiments the formulations can include one or more methylxanthine compounds. As used herein, the term “methylxanthine” refers to a compound classified as a methylated xanthine derivative, including, but not limited to, caffeine; theobromine; theophylline; aminophylline; doxofylline; pentoxifylline; 8-oxopentoxifylline; 8-oxolisofylline; and lisofylline.

Exemplary methylxanthines include caffeine; theophylline; 1-proparagyl 3,7-dimethyl xanthine; 7-proparagyl 1,3-dimethyl xanthine; 3-proparagyl 1,7-dimethyl xanthine; 1,3,7-triproparagyl xanthine; 3-isobutyl-1-methylxanthine (IBMX); 1,3,7-tripropyl xanthine; 7-benzyl-IBMX; 1-propyl 3,7-dimethyl xanthine; 1,3-dipropyl 7-methyl xanthine; 1,3-dipropyl 7-proparagyl xanthine; 3,7-dimethyl 1-propyl xanthine; and 7-ally 1,3-dimethyl xanthine. Various methylxanthines are well known in the art (see, for example, Daly et al., Pharmacol, 42:309-321 (1991); Ukena et al., Life Sci. 39:743-750 (1986); Choi et al., Life Sci. 43:387-398 (1988); Daly et al., J. Med. Chem. 29:1305-1308 (1986); Daly et al., Frog. Clin. Biol. Res. 230:41-63 (1987).

In some embodiments the formulation can contain one or more compounds having the general formula

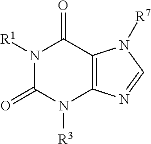

wherein R¹, R³, and R⁷ can be independently hydrogen, methyl, a halogen atom, a hydroxyl group, or any other organic groupings containing any number of carbon atoms and optionally including one or more heteroatoms such as oxygen, sulfur, or nitrogen grouping in linear, branched, or cyclic structural formats, representative R¹, R³, and R⁷ groupings being alkyl, substituted alkyl, propargyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C3-C20 cyclic, substituted C3-C20 cyclic, heterocyclic, substituted heterocyclic, aminoacid, peptide, or polypeptide group. In preferred embodiments, R¹, R³, and R⁷ independently can be hydrogen, methyl, ethyl, propyl, allyl, or propargyl. The term “propargyl” is defined as X—C≡C—CH₂—, wherein X is hydrogen, lower alkyl, haloalkyl, cycloalkyl, substituted or unsubstituted aryl, or substituted or unsubstituted heteroaryl.

In some embodiments, the formulations can contain one or more metabolites of caffeine, optionally in combination with caffeine. The term “metabolite”, as used herein, refers generally to any compound resulting from biotransformation of a parent compound after administration to an individual or organism, and includes forms of a compound comprising an additional chemical structure or moiety, or lacking a chemical structure or moiety present as a part of the parent compound prior to being contacted to an individual or an organism.

Metabolites of caffeine also contribute to caffeine's effects. Paraxanthine is responsible for an increase in the lipolysis process, which releases glycerol and fatty acids into the blood to be used as a source of fuel by the muscles. Theobromine is a vasodilator that increases the amount of oxygen and nutrient flow to the brain and muscles. Theophylline acts as a smooth muscle relaxant that chiefly affects bronchioles and acts as a chronotrope and inotrope that increases heart rate and force of contraction.

In some embodiments, the formulation can contain paraxanthine having the structure depicted below.

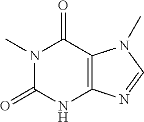

In some embodiments, the formulation can contain theobromine having the structure depicted below.

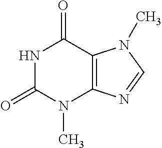

In some embodiments, the formulations can contain theophylline having the structure depicted below.

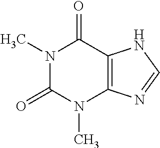

Caffeine is an achiral molecule without stereoisomers. In some embodiments the formulations can contain one or more analogs, derivatives, or metabolites of caffeine that exist as one or more stereoisomers. In such embodiments, the analog, derivative, or metabolite can be present as a single enantiomer, a purified composition containing essentially one or a few enantiomers, or a racemic mixture containing all or a few enantiomers.

The caffeine, caffeine analog, derivative, or metabolite, or salt thereof (jointly referred to herein as “caffeine unless noted otherwise) can be naturally occurring. Caffeine is synthesized in plants from the purine nucleotides adenosine monophosphate, guanosine monophosphate, and inosine monophosphate. Being readily available as a byproduct of decaffeination, caffeine is not usually synthesized chemically. Many naturally occurring analogs, derivatives, and analogs of caffeine can be plant derived as well. For example, theophylline is naturally found in high concentrations in cocoa beans. In some embodiments, the caffeine, caffeine analog, derivative, or metabolite can be non-naturally synthesized. For example, caffeine can be synthesized from dimethylurea and malonic acid.

B. Dosage Forms

Formulations disclosed herein are useful for controlled release of caffeine or a pharmaceutically acceptable salt, analog, derivative, or metabolite thereof. In certain embodiments, the formulation contains at least one extended release component and at least one rapid or immediate release component. In certain embodiments, the formulation contains more than two components, wherein each component has its own release profile. The immediate or rapid release component or components provide for an initial burst of the drug to produce effective in vivo concentrations. The extended release component or components maintain the effective in vivo concentrations of the drug for periods longer than would be observed using only an immediate or rapid release component.

In certain embodiments, the extended release component is the core of the dosage form wherein the drug is admixed with one or more excipients. In preferred embodiments, at least one of the excipients in the core is a release controlling polymer. In other embodiments, the extended release component contains a core and an additional layer which contains the drug and one or more excipients, which may include a release controlling polymer. The core may either be directly coated with the additional layer, or the core and additional layer may be separated by a layer containing a release controlling polymer which does not contain any drug. In further embodiments, the additional layer may be coated with a second additional layer containing the drug and one or more excipients, which may include a release controlling polymer. The second layer may be directly coated onto the first layer, or the first and second layers may be separated by a layer of a release controlling polymer that does not contain any drug.

Generally, the immediate or rapid release component is coated onto the extended release component. The immediate or rapid release component may be further coated with a protective layer which serves to maintain the integrity of the dosage form but does not substantially affect the release rate. The immediate or rapid release component may be a single layer of drug and excipient, or the immediate or rapid release component may be two or more layers, each of which are contain the drug and at least one excipient. Generally, an immediate or rapid release layer may also be designated as a “loading layer.”

In cases of multiple immediate or rapid release layers, one layer may be directly coated on top of another layer, or the two layers may be separated by a layer which does not contain any drug. If the non-drug containing layer contains one or more release controlling polymers, it is possible to deliver multiple boluses of drug as the formulation moves through the gastrointestinal tract.

In certain embodiments, the formulation contains a loading dose component, a delayed release component, and an extended release component. Each of the components is characterized by its own dissolution, and thus bioavailability, profile.

In some embodiments the formulation contains two loading dose components, delayed release component and an extended release component as a core. The outermost layer provides for immediate/rapid release of caffeine. The second loading dosage layer maintains plasma concentrations after the caffeine from the initial loading dose has been released.

In the preferred embodiment, the formulation is a tablet containing 250 to 500 mg caffeine, more preferably at least 400 mg caffeine. In certain embodiments, the formulation is capable of immediately releasing a bolus of drug followed by a sustained and/or delayed release of drug sufficient to maintain an in vivo blood plasma concentration between about 0.3 micrograms/mL and 40 micrograms/mL. The blood plasma concentration of caffeine may be determined using conventional techniques, including those employed for FDA-approved caffeine products. As used herein, blood plasma concentrations are in reference to an average value obtained from a representative cohort of individuals expected to metabolize caffeine normally.

Generally, the size of the formulation should be guided by general concerns of swallowability. In certain embodiments, the core tablet thickness is between about 3.5-5 mm, preferably between about 4-4.5 mm, and especially about 4.35 mm; the second loading dose film thickness is between 0.2-0.8 mm, preferably between 0.3-0.6 mm, and especially about 0.4 mm; the initial loading dose film thickness is between about 0.6-1.0 mm, preferably between about 0.7-0.8 mm, and especially about 0.75 mm. Other layers in the formulation may have thicknesses commensurate with those specified above.

Suitable oral dosage forms include tablets, capsules, nano, micro and particulate forms, and lozenges. Tablets can be made using granulation, compression or molding techniques well known in the art. Gelatin or non-gelatin capsules can prepared as hard or soft capsule shells, which can encapsulate liquid, solid, and semi-solid fill materials, using techniques well known in the art. For example, the drugs can be formulated into nanoparticles, microparticles, and combinations thereof, and encapsulated in a soft or hard gelatin or non-gelatin capsule. The particles can be formed of the drug and an extended release polymer or matrix. The capsule can be coated with one or more immediate release and/or rapid release dosing layers containing additional caffeine providing release of caffeine at certain times or at certain locations along the gastrointestinal tract. The rapid release dosing layers can optionally have a delayed release or be coated with a delayed release polymer coating.

Extended Release Components

The extended release components are prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. Materials used in the preparation of extended release components include insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such ashydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including, but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers. In certain preferred embodiments, the acrylic polymer is comprised of one or more ammonio methacrylate copolymers Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin such as that which is commercially available from Rohm Pharma under the tradename EUDRAGIT®. In further preferred embodiments, the acrylic polymer comprises a mixture of two acrylic resins commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers such as EUDRAGIT® RL/RS may be mixed together in any desired ratio in order to ultimately obtain an extended-release component having a desirable dissolution profile. Desirable sustained-release multiparticulate systems may be obtained, for instance, from 100% EUDRAGIT®, 50% EUDRAGIT® RL and 50% EUDRAGIT® RS, and 10% EUDRAGIT® RL and 90%: EUDRAGIT® 90% RS. One skilled in the art will recognize that other acrylic polymers may also be used, such as, for example, EUDRAGIT® L.

Alternatively, extended release components can be prepared using osmotic systems or by applying a semi-permeable coating to the dosage form. In the latter case, the desired drug release profile can be achieved by combining low permeable and high permeable coating materials in suitable proportion.

In another embodiment, the caffeine is dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

Delayed Release Components

Delayed release formulations can be created by coating a solid dosage form with a polymer film, which is insoluble in the acidic environment of the stomach, and soluble in the neutral environment of the small intestine.

pH dependent polymers are frequently used to delay release, for example following ingestion, until the composition has passed through the low pH of the stomach and entered into the higher pH of the small intestine. Representative pH dependent polymers include, but not limited to, methyl acrylate-methacrylic acid copolymers, cellulose acetate succinate, hydroxypropyl methylcellulose phthalate, hydroxypropyl methylcellulose acetate succinate (hypromellose acetate succinate), polyvinyl acetate phthalate, methyl methacrylate-methacrylic acid copolymers, and sodium alginate. Fatty acids, such as stearic acid, and salts thereof may also be employed in delayed release formulations.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon.

Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITs NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, dosage form and route of administration that produces the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Rapid Release and Immediate Release Components

One or more loading dosages can be prepared by creating coated or multilayer coated capsules or tablets, i.e. a tablet for incorporation into a capsule, a tablet for use as an inner core in a “coated core” dosage form, or a plurality of drug-containing beads, particles or granules, for incorporation into either a tablet or capsule. Coated capsules and tablets can be prepared with a loading dosage by applying or spraying a thin film of a biocompatible polymer containing the loading dosage of caffeine or other active agent, thereby creating a loading dose layer. Suitable film-forming polymers include both synthetic and natural polymers such as polyvinylpyrrolidone, polyvinyl alcohol, partially hydrolysed polyvinyl acetate, modified polyvinylpyrrolidone such as a polyvinylpyrrolidone/vinyl acetate copolymer, polyethylene oxides, ethylene/maleic anhydride copolymer, methyl vinyl ether-maleic anhydride copolymer, water-soluble cellulose such as carboxymethylcellulose, water-soluble polyamides or polyesters, copolymers and homopolymers of acrylic acids, starches, natural gums such as alginates, dextrins and proteins such as gelatins and caseins. Mixtures of such film-forming polymers may also be used. The rate of dissolution will depend on a number of factors, in some cases including the particular nature of the film-forming polymer and the encapsulated material.

In some embodiments, formulations can contain more than one loading dosage, such as a first loading dosage followed by a second delayed loading dosage.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Additional Components

Formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Coatings may be formed with a different ratio of water soluble polymer, water insoluble polymers and/or pH dependent polymers, with or without water insoluble/water soluble non polymeric excipient, to produce the desired release profile. The coating is either performed on dosage form (matrix or simple) which includes, but not limited to, tablets (compressed with or without coated beads), capsules (with or without coated beads), beads, particle compositions, “ingredient as is” formulated as, but not limited to, suspension form or as a sprinkle dosage form.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to antioxidants such as butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

In the most preferred embodiment, the core tablet thickness is 4.35 mm; the first layer thickness is 0.4 mm; the second layer thickness is 0.75 mm, and the total tablet weight 850 mg.

The loading dose layers can be of a variety of thicknesses depending upon the nature of the film-forming polymer, the presence or absence of additional additives, and the amount of caffeine in the loading dose layer. The loading dose layer can have a thickness of up to about 5 mm, up to about 4.5 mm, up to about 3.5 mm, up to about 3 mm, up to about 2 mm, up to about 1 mm, up to about 0.6 mm, up to about 0.3 mm.

In the controlled release caffeine formulations containing extended release tablets having one or more loading dosage layers, the amount of the caffeine or caffeine analog, derivative, or metabolite will generally be distributed about equally between the extended release core and the loading dosage layers, i.e. the extended release core will generally contain from about 20% to about 70%, preferably from about 30% to about 60% of the total dosage with the remainder, constituting a loading dosage, being distributed in the one or more loading dosage layers. When there is more than one loading dosage layer, the outermost/first-most dosing layer will generally contain the majority of the initial dosage amount. In some embodiments, the outermost/first-most initial dosing layer contains from about 60% to about 90%, from about 70% to about 80% of the loading dosage amount. As a non-limiting example, in a coated tablet form having two loading dosage layers and a total loading dosage of 250 mg of caffeine, the outermost/first-most loading dosage layer could contain 200 mg with the second loading dosage layer containing 50 mg. In such a tablet, the extended release core contains 150 mg caffeine to yield a dosage form having 400 mg total caffeine.

### III. METHODS OF MANUFACTURE

The core extended release caffeine tablets can be manufactured by readily available equipment, and the one or more coating layers containing pH dependent polymers, film forming polymers, caffeine, and other additives can be applied by readily available equipment that can be integrated into widely-used pharmaceutical processes. Among other methods, direct compression is particularly useful for the formation of the core, and direct coating methods may be employed for applying the additional layers to the core.

Extended release tablets containing wax materials are generally prepared using methods known in the art such as a direct blend method, a congealing method, and an aqueous dispersion method. In the congealing method, the drug is mixed with a wax material and either spray—congealed or congealed and screened and processed.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. Rather than selecting a particular surface for coating, the present formulations allows for coating the whole tablet at the same time providing different drug release profiles from different layers.

### IV. METHODS OF USE

Controlled release dosage formulations capable of quickly reaching an initial peak blood concentration and maintaining a minimum effective caffeine level over an extended period of time, thereby eliminating the need for follow up doses, are administered to an individual in need thereof to maintain useful levels of caffeine over a period of up to 10 hours, up to 8 hours, or up to 6 hours. The formulations are capable in some embodiments of attaining peak blood plasma concentrations in a period similar to dietary caffeine or similar to immediate-release caffeine formulations. In some embodiments, the initial peak blood plasma concentration is reached within 2 hours of ingestion, within one hour of ingestion, or within 45 minutes of ingestion. In some embodiments, the initial peak blood plasma concentration is at least about 10 μg/ml, at least about 20 μg/ml, at least about 30 μg/ml as compared to the baseline levels. In some embodiments, the initial peak blood plasma concentration is less than about 40 μg/ml, less than about 35 μg/ml, or less than about 30 μg/ml as compared to the baseline levels. In some embodiments the maximum blood plasma concentration (Cₘₐₓ) is less than that obtained with other caffeine formulations without a concomitant reduction in the area under the curve (AUC) and without extension of the time in which the maximum plasma concentration is obtained (T.).

The controlled release caffeine formulations can maintain a minimum blood plasma concentration for a period of time that can be 4 hours, up to about 6 hours, up to about 8 hours, or up to about 10 hours. In preferred embodiments, the formulations can maintain a blood plasma concentration between about 0.1 μg/ml and about 80 μg/ml above baseline levels, preferably between about 10 μg/ml and about 40 μg/ml, and more preferably between about 20 μg/ml and about 30 μg/ml above baseline levels for a period of at least 4 hours, at least 6 hours, or at least 10 hours after the initial peak blood plasma concentration. Although it is generally preferred that plasma concentrations not exceed 80 μg/ml, one of ordinary skill will appreciate that some individuals may briefly experience blood plasma concentrations higher than 80 μg/mL. In such cases, it is preferred that the 80 μg/mL threshold concentration not be exceeded for periods greater than 10 minutes, preferably, 5 minutes, and even more preferably no more than 1 minute.

In one embodiment, the formulations release between about 25-70% of the caffeine or pharmaceutically acceptable salt, analog, derivative or metabolite thereof (i.e., the drug) within about 30 minutes to about three hours after administration of the formulation. In other embodiments, between about 40-60% of the drug is released over this time period, and in preferred embodiments, between about 45-55% of the drug is released over this time period. In an especially preferred embodiment, about 50% of the drug is released within about 30 minutes to about 3 hours after administration of the formulation.

In other embodiments, about 75% of the drug is released within about 4 hours of administration of the formulation, and preferably about 70% is released. In an especially preferred embodiment, about 65% of the drug is released within about 4 hours of administration of the formulation.

In other embodiments, about 90% of the drug is released within about 5 hours after administration of the formulation, and preferably about 85% is released. In an especially preferred embodiment, about 80% of the drug is released within about 5 hours after administration of the formulation.

In other embodiments, about 95% of the drug is released within about 6 hours after administration of the formulation and about 100% of the drug is released within about 7 hours after administration of the formulation.

In a particularly preferred embodiment, the formulation releases about 50% of the drug within about 30 minutes to about 3 hours after administration of the formulation, about 65% of the drug within about 4 hours after administration of the formulation, about 80% of the drug within about 5 hours after administration of the formulation, about 95% of the drug within about 6 hours after administration of the formulation, and about 100% of the drug within about 7 hours after administration of the formulation. In especially preferred embodiments, the drug is caffeine or a pharmaceutically acceptable salt thereof.

### V. EXAMPLES

The present invention will be further understood by reference to the following non-limiting examples.

### Example 1

### 400 mg Caffeine Controlled Release Tablets

| | |
|---|---|
| Ingredient | Amount (mg) |

Core

| | |
|---|---|
| Caffeine | 150 |
| Microcrystalline cellulose | 116 |
| Hydroxypropyl methycellulose K100LV CR | 20 |
| Hydroxypropyl methycellulose E5 | 20 |
| Silicon dioxide | 8 |
| Magnesium stearate | 6 |

Coating I

| | |
|---|---|
| EUDRAGIT ® L100 | 45 |

First layer

| | |
|---|---|
| Caffeine | 50 |
| Polyvinylpyrrolidone | 25 |

Coating II

| | |
|---|---|
| EUDRAGIT ® L100-55 | 45 |

Second layer

| | |
|---|---|
| Caffeine | 200 |
| Polyvinylpyrrolidone | 100 |
| Polysorbate 80 | 12 |

### Example 2

### Caffeine Controlled Release Tablets, 400 mg

| | |
|---|---|
| Ingredient | Amount (mg) |

Core

| | |
|---|---|
| Caffeine | 150 |
| Microcrystalline cellulose | 166 |
| Hydroxypropyl methycellulose K100LV CR | 20 |
| Hydroxypropyl methycellulose E5 | 30 |
| Silicon dioxide | 8 |
| Magnesium stearate | 6 |

First layer

| | |
|---|---|
| Caffeine | 50 |
| Polyvinylpyrrolidone | 25 |

Coating I

| | |
|---|---|
| EUDRAGIT ® L100 | 45 |

Second layer

| | |
|---|---|
| Caffeine | 200 |
| Polyvinylpyrrolidone | 100 |
| Polysorbate 80 | 12 |

### Example 3

### Caffeine Controlled Release Tablets, 400 mg

| | |
|---|---|
| Ingredient | Amount (mg) |

Core

| | |
|---|---|
| Caffeine | 200 |
| Microcrystalline cellulose | 50 |
| EUDRAGIT ® L100 | 100 |
| Cetyl alcohol | 66 |
| Crospovidone | 20 |
| Silicon dioxide | 8 |
| Magnesium stearate | 6 |

Outer layer

| | |
|---|---|
| Caffeine | 200 |
| Polyvinylpyrrolidone | 100 |
| Polysorbate 80 | 12 |

### Example 4

### Additional Caffeine Controlled Release Tablets, 400 mg

| Batch No's | | |
|---|---|---|
| 52-180/D | 52-145C/D | 52-132D/F |

| | |
|---|---|
| Ingredients | Qty/Tab in mg |

Core Tablet:

| | | | |
|---|---|---|---|
| Caffeine (Anhydrous powder) | 150.00 | 150.00 | 150.00 |
| Microcrystalline Cellulose 200 NF | 166.00 | 166.00 | 166.00 |
| (Avicel PH 200) | | | |
| HPMC (Methocel K100 | 20.00 | 20.00 | 20.00 |
| premium LVCR) | | | |
| HPMC (Methocel E5 premium | 30.00 | 30.00 | 30.00 |
| LV) | | | |
| Crospovidone USP (Kollidon CL) | 20.00 | 20.00 | 20.00 |
| Colloidal Silicon dioxide, NF | 8.00 | 8.00 | 8.00 |
| Magnesium Stearate, NF | 6.00 | 6.00 | 6.00 |

Drug Coating

| | | | |
|---|---|---|---|
| Caffeine (Anhydrous powder) | 50.00 | 50.00 | 50.00 |
| Hydroxy propylcellulose (Klucel | 25.00 | 25.00 | 25.00 |
| LF), NF | | | |
| Iso propyl Alcohol, USP 99% | q.s | q.s | q.s |
| ACS grade | | | |
| Purified water USP | q.s | q.s | q.s |

Enteric coating

| | | | |
|---|---|---|---|
| Methacrylic acid Copolymer | 40.00 | 40.00 | 40.00 |
| (Eudragit L100) | | | |
| Triethyl citrate PG/NF | 4.00 | 4.00 | 4.00 |
| Iso propyl Alcohol, USP 99% | q.s | q.s | q.s |
| ACS grade | | | |

IR drug coating

| | | | |
|---|---|---|---|
| Caffeine (Anhydrous powder) | 200.00 | 200.00 | 200.00 |
| Povidone K-30, USP | 50.00 | 50.00 | 50.00 |
| Polysorbate 80, NF (Tween 80) | 12.00 | 12.00 | 12.00 |
| Sodium Lauryl sulfate, NF | 8.00 | 8.00 | 8.00 |
| Iso propyl Alcohol, USP 99% | q.s | q.s | q.s |
| ACS grade | | | |
| Purified water USP | q.s | q.s | q.s |
| | 789.00 | 789.00 | 789.00 |

Film Coating over drug coated tablets

| | | | |
|---|---|---|---|
| Drug coated tablets | 789.00 | — | 789.00 |
| Opadry clear YS-3-19024 | 20.00 | | — |
| Opadry II 85F13932 Orange | — | — | 30.00 |
| Total wt of tablet in mg | 809.00 | — | 819.00 |

| | |
|---|---|
| | Batch No |
| | 52-98AE/D |
| Ingredients | Qty/Tab in mg |

Core Tablet

| | |
|---|---|
| Caffeine (Anhydrous powder) | 200.00 |
| Microcrystalline Cellulose 200 NF | 116.00 |
| (Avicel PH 200) | |
| HPMC (Methocel K100 premium | 30.00 |
| LVCR) | |
| HPMC (Methocel E5 premium LV) | 20.00 |
| Crospovidone USP (Kollidon CL) | 20.00 |
| Colloidal Silicon dioxide, NF | 8.00 |
| Magnesium Stearate, NF | 6.00 |
| Wt of tablet in mg | 400.00 |

Enteric Coating

| | |
|---|---|
| Methacrylic acid Copolymer (Eudragit | 40.00 |
| L100) | |
| Polyethylene Glycol 400 (PEG 400) | 4.00 |
| Iso propyl Alcohol, USP 99% ACS grade | q.s |

IR drug coating

| Caffeine (Anhydrous powder) | 200.00 |
|---|---|
| Hydroxy propylcellulose (Klucel LF), NF | 100.00 |
| Iso propyl Alcohol, USP 99% ACS grade | q.s |
| Purified water USP | q.s |
| Total wt of tablet in mg | 744.00 |
| | Batch No's |
| | 52/104AE/D |
| Ingredients | Qty/Tab in mg |

Core Tablet

| | |
|---|---|
| Caffeine (Anhydrous powder) | 150.00 |
| Microcrystalline Cellulose 200 NF | 116.00 |
| (Avicel PH 200) | |
| HPMC (Methocel K100 premium | 20.00 |
| LVCR) | |
| HPMC (Methocel E5 premium LV) | 30.00 |
| Crospovidone USP (Kollidon CL) | 20.00 |
| Colloidal Silicon dioxide, NF | 8.00 |
| Magnesium Stearate, NF | 6.00 |
| Wt of tablet in mg | 350.00 |

Enteric Coating

| | |
|---|---|
| Methacrylic acid Copolymer (Eudragit | 40.00 |
| L100) | |
| Polyethylene Glycol 400 (PEG 400) | 4.00 |
| Iso propyl Alcohol, USP 99% ACS grade | q.s |

IR drug coating

| | |
|---|---|
| Caffeine (Anhydrous powder) | 50.00 |
| HPMC (Methocel E5 premium LV) | 25.00 |
| Iso propyl Alcohol, USP 99% ACS grade | q.s |
| Purified water USP | q.s |
| Wt of tablet in mg | 469.00 |

Enteric Coating

| | |
|---|---|
| Methacrylic acid Copolymer (Eudragit | 40.00 |
| L100) | |
| Polyethylene Glycol 400 (PEG 400) | 4.00 |
| Iso propyl Alcohol, USP 99% ACS grade | q.s |
| Total wt of tablet in mg | 513.0 |

In the formulations of Example 4, the core is prepared by direct compression, while each of the subsequent layers are applied using a conventional coating apparatus.

### Example 5

### Release Profile of Exemplary Caffeine Controlled Release Tablets

Dissolution tests were performed on 400 mg caffeine controlled release tablets using a 900 ml basket at 100 rpm in 0.1N HCl for 2 hours, followed by pH 4.5 acetate buffer for one hour, followed by 7 hours in pH 6.8 Phosphate buffer.

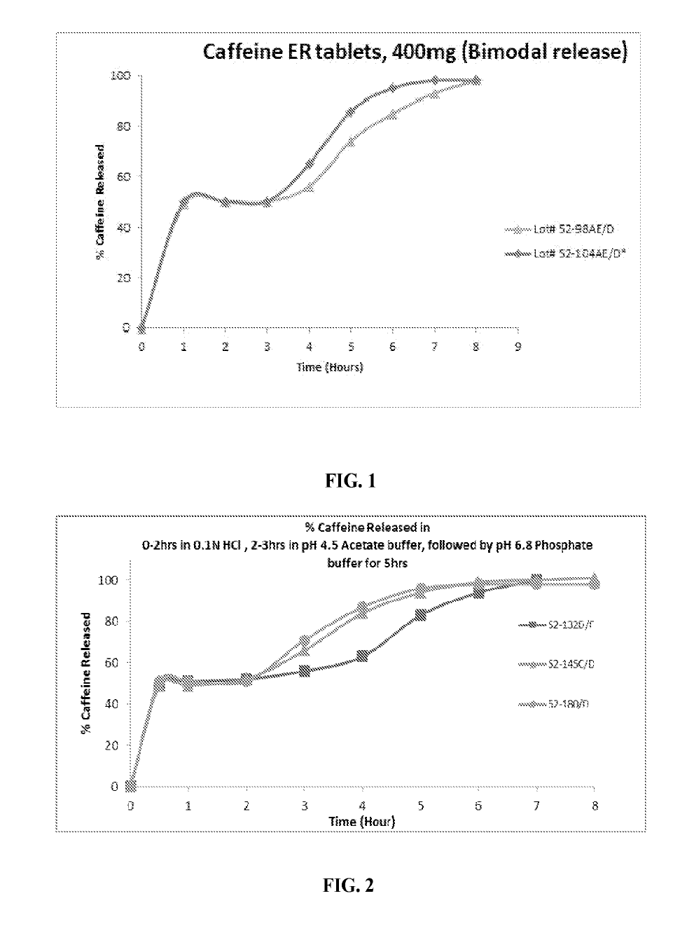

Results for two formulations are shown in Tables 1 and 2. The results are also graphed in FIGS. 1 and 2.

TABLE 1
Dissolution results for exemplary 400 mg caffeine
controlled release tablets 52-104AE/D

| Time | % Caffeine Released |
|---|---|
| (hours) | Tablet 52-104AE/D |
| 0 | 0 |
| 1 | 45 |
| 2 | 48 |
| 3 | 50 |
| 4 | 65 |
| 5 | 86 |
| 6 | 95 |
| 7 | 98 |
| 8 | 98 |

TABLE 2
Dissolution results for exemplary 400 mg caffeine controlled
release tablets 52-132D/F; 52-145C/D, and 52/180/D

| | % Drug Released | | |
|---|---|---|---|
| | Batch Number | | |
| Time (hour) | 52-132D/F | 52-145C/D | 52-180/D |
| 0 | 0 | 0 | 0 |
| 0.5 | 49 | 49 | 51 |
| 1 | 51 | 49 | 51 |
| 2 | 52 | 52 | 51 |
| 3 | 56 | 66 | 71 |
| 4 | 63 | 84 | 87 |
| 5 | 83 | 94 | 96 |
| 6 | 94 | 99 | 98 |
| 7 | 100 | 100 | 98 |
| 8 | 102 | 101 | 98 |

### Example 6

### Additional Formulations

The following formulations are further embodiments:

| | mg/Tablet | | | | | | |
|---|---|---|---|---|---|---|---|
| | Batch: 052- | Batch: 052- | Batch: 052- | Batch: 052- | Batch: 052- | Batch: 052- | Batch: 052- |
| Ingredient | 153 | 155 | 156 | 159 | 161 | 176 | 177 |

ER Portion

| | | | | | | | |
|---|---|---|---|---|---|---|---|
| Caffeine | 250.00 | 250.00 | 250.00 | 250.00 | 250.00 | 250.00 | 250.00 |
| (Anhydrous | | | | | | | |
| Powder) | | | | | | | |
| Microcrystalline | 170.00 | 170.00 | 170.00 | 170.00 | 170.00 | 155.00 | 155.00 |
| Cellulose, NF | | | | | | | |
| (Avicel PH | | | | | | | |
| 200) | | | | | | | |
| Methocel K 100 | 42.00 | 32.00 | 32.00 | 22.00 | 20.00 | 10.00 | 20.00 |
| M Premium | | | | | | | |
| Methocel K 100 | 20.00 | 30.00 | 20.00 | 30.00 | 40.00 | 35.00 | 25.00 |
| Premium LVCR | | | | | | | |
| Eudragit L 100 | — | — | — | — | — | 40.00 | 40.00 |
| FD&C Red # 40 | | — | — | — | 0.25 | 0.25 | 0.25 |
| Al. Lake (15-17%) | | | | | | | |
| Colloidal | 10.00 | 10.00 | 10.00 | 10.00 | 5.00 | 5.00 | 5.00 |
| Silicon dioxide, | | | | | | | |
| NF | | | | | | | |
| Magnesium | 8.00 | 8.00 | 8.00 | 8.00 | 4.75 | 4.75 | 4.75 |
| Stearate, NF | | | | | | | |
| ER Portion | 500.00 | 500.00 | 490.00 | 490.00 | 490.00 | 500.00 | 500.00 |
| Tablet Weight | | | | | | | |

IR Portion

| | | | | | | | |
|---|---|---|---|---|---|---|---|
| Caffeine | 150.00 | 150.00 | 150.00 | 150.00 | 150.00 | 150.00 | 150.00 |
| (Anhydrous | | | | | | | |
| Powder) | | | | | | | |
| Microcrystalline | 90.00 | 90.00 | 90.00 | 90.00 | 90.00 | 90.00 | 90.00 |
| Cellulose, NF | | | | | | | |
| (Vivapur Type | | | | | | | |
| 102) | | | | | | | |
| Povidone K 30 | 10.00 | 10.00 | 10.00 | 10.00 | 10.00 | 10.00 | 10.00 |
| Crospovidone, | 12.00 | 12.00 | 12.00 | 12.00 | 12.00 | 12.00 | 12.00 |
| NF | | | | | | | |
| Colloidal | 8.00 | 8.00 | 8.00 | 8.00 | 8.00 | 8.00 | 8.00 |
| Silicon dioxide, | | | | | | | |
| NF | | | | | | | |
| Magnesium | 5.00 | 5.00 | 5.00 | 5.00 | 5.00 | 5.00 | 5.00 |
| Stearate, NF | | | | | | | |
| IR Portion | 275.00 | 275.00 | 275.00 | 275.00 | 275.00 | 275.00 | 275.00 |
| Tablet Weight | | | | | | | |
| Total Tablet | 775.00 | 775.00 | 765.00 | 765.00 | 765.00 | 775.00 | 775.00 |
| Weight | | | | | | | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims. It will be understood that the embodiments of the present invention that are described are merely exemplary and that a person skilled in the art can make many variations to these embodiment

1. An oral formulation comprising:
a first component providing for immediate or rapid release of caffeine, and
a second component providing for extended release of caffeine,
wherein the formulation provides a blood plasma concentration of caffeine at least about 0.3 μg/mL and maintains the concentration for at least four hours.

2. The formulation of claim 1 comprising at least about 250 mg.

3. The formulation of claim 1 comprising at least about 400 mg caffeine.

4. The formulation according to claim 1, wherein the formulation provides a blood plasma concentration of caffeine is at least about 0.3 μg/mL for at least about six hours.

5. The formulation of claim 4, wherein the first component releases a caffeine bolus, and wherein the second component releases caffeine such that administration of the formulation produces an in vivo blood plasma concentration of caffeine between about 0.3 μg/mL and about 80 μg/mL for at least six hours.

6. The formulation of claim 5, wherein the formulation produces an in vivo blood plasma concentration of caffeine between about 0.3 μg/mL and about 80 μg/mL for at least eight hours.

7. The formulation according to claim 1, wherein after oral administration about 25-70% of the caffeine is released within about 30 minutes to about 3 hours post-administration, at least about 75% of the caffeine is released within about 4 hours post-administration, at least about 90% of the caffeine is released within about 5 hours post-administration, at least about 95% of the caffeine is released within about 6 hours post-administration, and about 100% of the caffeine is released within about 7 hours post-administration.

8. The formulation of claim 7, wherein the release is measured by a dissolution test performed in 0.1N HCl for 2 hours, in pH 5.5 acetate buffer between 2-3 hours, and pH 6.8 phosphate buffer between 3-8 hours.

9. The formulation of claim 1, wherein the first component provides for an immediate release of up to about 50% of the total caffeine within about one hour after administration under physiological conditions, and wherein the second component provides release of the remaining caffeine that is delayed until at least 3 hours after administration, and release occurs up to about 8 hours after administration under physiological conditions.

10. The formulation of claim 9, wherein the second component provides release of the remaining caffeine that is delayed until at least 4 hours after administration

11. The formulation of claim 1, wherein the extended release component comprises a core comprising caffeine.

12. The formulation of claim 11, wherein the extended release component comprises one or more hydrophilic polymers.

13. The formulation of claim 12, wherein the one or more hydrophilic polymers are selected from the group consisting of cellulosic polymers such as methyl and ethyl cellulose, hydroxyalkylcelluloses such as hydroxypropyl-cellulose, hydroxypropylmethylcellulose, sodium carboxymethylcellulose, polyethylene glycols, polyethylene oxides and mixtures thereof.

14. The formulation of claim 11, wherein the extended release component further comprises a layer comprising caffeine and at least one release controlling polymer.

15. The formulation of claim 14, wherein the core and the layer are separated by a coating that does not contain caffeine.

16. The formulation of claim 14, wherein the coatings comprise one or more polymers independently selected from the group consisting of bioerodible polymers, hydrolysable polymers, gradually water soluble polymers, enzymatically degradable polymers, and enteric polymers.

17. The formulation of claim 16, wherein the one or more polymers selected from the group consisting of polyethylene glycols, polyethylene oxides, hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate, carboxymethylcellulose sodium; acrylic acid polymers, methacrylic resins, vinyl polymers, polyvinyl pyrrolidone, vinyl acetate, vinylacetate phthalate, vinylacetate crotonic acid copolymer, ethylene-vinyl acetate copolymer, azo polymers, pectin, chitosan, amylose and guar gum, zein and shellac.

18. The formulation of claim 1, comprising:
a first loading dosage;
a second loading dosage;
an extended release core,
wherein each loading dosage and core comprise caffeine; and
a film-forming polymer

19. The formulation of claim 18, wherein the loading dosages in combination contain about 250 mg of caffeine,
wherein one loading dosage comprises about 200 mg of caffeine, and
wherein the second loading dosage comprises about 50 mg of caffeine.

20. The formulation of claim 19, wherein the first loading dosage component releases within one hour under physiological conditions.

21. The formulation of claim 19, wherein the extended release core does not release caffeine in pH below 5.5.

22. The formulation of claim 18, wherein the loading dosage is coated with at least one film-forming polymer.

23. The formulation of claim 22, wherein the film-forming polymer is selected from the group consisting of polyvinylpyrrolidone, polyvinyl alcohol, partially hydrolysed polyvinyl acetate, modified polyvinylpyrrolidone such as a polyvinylpyrrolidone/vinyl acetate copolymer, polyethylene oxides, ethylene/maleic anhydride copolymer, methyl vinyl ether-maleic anhydride copolymer, water-soluble cellulose such as carboxymethylcellulose, water-soluble polyamides or polyesters, copolymers and homopolymers of acrylic acids, starches, natural gums such as alginates, dextrins and proteins such as gelatins and caseins.

24. The formulations of claim 1, wherein the caffeine is caffeine free base or a pharmaceutically acceptable salt, analog, derivative or metabolite thereof.

25. A method of administering the formulation of claim 1 to an individual in need thereof comprising orally administering the formulation.

26. The method of claim 25, wherein formulation is administered to produce a peak blood plasma concentration of caffeine between about a formulation immediately releasing a caffeine bolus followed by a sustained and/or delayed release of caffeine to maintain an in vivo blood plasma concentration of caffeine between 0.3 micrograms/mL and 40 micrograms/mL.

27. The method of claim 26, wherein the blood plasma concentration of caffeine is maintained between about 10 micrograms/ml and about 30 micrograms/ml for up to about six hours after reaching the initial peak blood plasma concentration.

28. The method of claim 26, wherein the blood plasma concentration of caffeine is maintained between about 20 micrograms/ml and about 40 micrograms/ml for up to about six hours after reaching the initial peak blood plasma concentration.

29. The method of claim 26, wherein the initial peak blood plasma concentration is reached within about one hour after administration.
